(19) United States
(12) Patent Application Publication
(10) Pub. No.: US 2017/0029388 A1
(43) Pub. Date: Feb. 2, 2017

(54) Treatment of Neurodegenerative Diseases with Asparagine Endopeptidase (AEP) Inhibitors and Compositions Related Thereto

(71) Applicant: INTERCONTINENTAL GREAT BRANDS LLC, East Hanover, NJ (US)

(72) Inventors: Keqiang Ye, Lilburn, GA (US); Haian Fu, Decatur, GA (US); Yuhong Du, Atlanta, GA (US)

(21) Appl. No.: 15/303,458

(22) PCT Filed: Apr. 8, 2015
(86) PCT No.: PCT/US2015/024864

§ 371 Date: Oct. 11, 2016

Related U.S. Application Data
(60) Provisional application No. 61/978,362, filed on Apr. 11, 2014.

(51) Int. Cl. C07D 271/12

(52) U.S. Cl. CPC C07D 271/12

(57) ABSTRACT

This disclosure relates to asparagine endopeptidase inhibitors and compositions and uses related thereto. In certain embodiments, the asparagine endopeptidase inhibitors are useful for treating or preventing neurodegenerative diseases and cognitive disorders such as Alzheimer's Disease. In certain embodiments, the disclosure relates to pharmaceutical compositions comprising an asparagine endopeptidase inhibitor and a pharmaceutically acceptable excipient. In certain embodiments, the disclosure relates to methods of treating or preventing a neurodegenerative disease comprising administering an effective amount of pharmaceutical composition a asparagine endopeptidase inhibitor disclosed herein to a subject in need thereof.

### CROSS REFERENCE TO RELATED APPLICATIONS

This Application claims priority to U.S. Provisional Application No. 61/978,362 filed Apr. 11, 2014, hereby incorporated by reference in its entirety.

### ACKNOWLEDGEMENT

This invention was made with government support under Grant RO1NS060680 awarded by the National Institutes of Health. The Government has certain rights in the invention.

### BACKGROUND

Asparagine endopeptidase (AEP), also known as legumain, is a lysosomal cysteine protease that cleaves peptide bonds C-terminally to asparagine residues. AEP is involved in various cellular events, including antigen processing, the cleavage of other lysosomal enzymes, osteoclast formation, and proper kidney functionality. In mammals, AEP is highly expressed in the kidneys; mice deficient in AEP accumulate various proteins in the endosomes and lysosomes of the proximal tubule cells of their kidneys, which results in a pathology consisting of hyperplasia, fibrosis and glomerular cysts. AEP-null mice exhibit symptoms similar to those of hemophagocytic lymphohistiocytosis, suggesting the enzyme is involved in the pathophysiology of this disease. Biochemically, the enzyme is highly regulated by its specificity for asparagine residues and pH. The particular motif that AEP uses to recognize its substrates is not completely understood. Dysregulated AEP activity has been implicated in various diseases, including cancers and neurodegeneration. See Basurto-Islas et al., Activation of asparaginyl endopeptidase leads to tau hyperphosphorylation in Alzheimer disease, J Biol Chem 288, 2013, 17495-17507. Chan et al., Mice lacking asparaginyl endopeptidase develop disorders resembling hemophagocytic syndrome, Proc Natl Acad Sci USA, 2009, 106, 468-473. Herskowitz et al., Asparaginyl endopeptidase cleaves TDP-43 in brain, Proteomics, 2012, 12, 2455-2463.

Ovat et al. report aza-peptidyl Michael acceptor and epoxide inhibitors as inhibitors of Schistosoma mansoni and Ixodes ricinus legumains (asparaginyl endopeptidases). See J Med Chem, 2009, 52, 7192-7210.

Loak et al. report acyloxymethylketone inhibitors of asparaginyl endopeptidase. See Biol Chem, 2003, 384, 1239-1246.

Niestroj et al. report inhibition of mammalian legumain by Michael acceptors and AzaAsn-halomethylketones. See Biol Chem (2002) 383, 1205-1214.

Xiang et al. report DNA vaccines target the tumor vasculature and microenvironment and suppress tumor growth and metastasis. See Immunol Rev, 2008, 222, 117-128.

References cited herein are not an admission of prior art.

### SUMMARY

This disclosure relates to asparagine endopeptidase inhibitors and compositions and uses related thereto. In certain embodiments, the asparagine endopeptidase inhibitors are useful for treating or preventing neurodegenerative diseases and cognitive disorders such as Alzheimer's Disease. In certain embodiments, the disclosure relates to pharmaceutical compositions comprising an asparagine endopeptidase inhibitor and a pharmaceutically acceptable excipient. In certain embodiments, the disclosure relates to methods of treating or preventing a neurodegenerative disease comprising administering an effective amount of pharmaceutical composition a asparagine endopeptidase inhibitor disclosed herein to a subject in need thereof.

### BRIEF DESCRIPTION OF THE FIGURES

FIG. 1 illustrates a high-throughput screening scheme. An Asinex library of 54,384 compounds was screened with mouse kidney lysates, then counter-screened with AEP knock-out lysates to yield 736 hits with IC₅₀ values less than or equal to 40 μM. The hits were validated further with purified human AEP, and promising compounds were categorized into 8 groups. Compounds from each group were tested and the cytotoxicity and specificity were determined.

FIG. 2 shows data on the determination of IC₅₀ values. Purified recombinant enzyme was incubated with various concentrations of inhibitor in appropriate assay buffers in the presence of increasing concentrations of inhibitor. The formation of fluorescent product was monitored in duplicate reactions and the data was fit to appropriate equations to calculate the IC₅₀ values.

FIG. 3A-H shows data on the determination of IC₅₀ values in intact Pala cells. The cells were incubated with inhibitors for 2 hrs then cells washed, harvested and lysed and the residual enzymatic activity was determined. Lysate was normalized by Bradford assay and the experiment was performed in triplicate and the mean results and SEM were plotted.

FIG. 4A-D shows data on cytotoxicity and genotoxicity of compounds. A. MTT assay, in which 50 μM of each compound was incubated with HepG2 cells for 24 hrs. The compound-containing medium was removed and the cells were incubated with MTT solution for 3 hrs. Subsequently, the MTT solution was replaced with DMSO and the 0D₅₇₀ was observed. B. LDH assay, in which 50 μM of each compound was incubated with primary culture neurons for 48 hrs. The media was then collected and incubated with LDH assay substrate for 30 min at room temp, in the dark. After the reactions were quenched, the 0D490 was observed. C. COMET assay results; 50 μM compound was incubated with HepG2 cells for 24 hrs. The cells were then added low-melt agarose and plated on microscope slides. The cells were lysed and the DNA was denatured and subject to electrophoresis. Finally, the DNA was stained with SYBR Green and 100 nuclei were counted for each sample, in each experiment; the experiment was performed in triplicate. D. Micronucleus assay results; 50 μM compound was incubated with HepG2 cells for 24 hrs. Cells were fixed and nuclei were stained with DAPI and 1,000 cells were counted per sample; three independent experiments were performed.

FIG. 5 shows data indicating DTT Reversibility. AEP was reacted with specified inhibitor, after 15 min 10 mM DTT or L-cysteine was added to the reaction and the fluorescent signal was read for an additional 15 min. At the end of the second 15 min incubation, the percentage of product formed in the presence of each compound was determined in comparison to the DMSO control reaction.

FIG. 6A-H shows data on competitive, slow-binding inhibitors of AEP. Steady-state kinetic parameters were determined from Michaelis-Menten plots, fit to a competitive inhibition equation, by varying substrate, Z-AAN-AMC, at fixed concentrations of: A. Compound 11 B. Compound BB1. KI values for each inhibitor were determined by global fits to the competitive inhibition equation. C. Time course inactivation assays were used to generate progress curves, depicting product formation as a function of time. Pseudo first-order rate constants were obtained at each concentration of compound 11. D. Re-plot of the pseudo first-order rate constants, k_{obs}, vs. the concentration of compound 11. E. Time course inactivation assays were used to generate progress curves, depicting product formation as a function of time. Pseudo first-order rate constants were obtained at each concentration of compound BB1. F. Re-plot of the pseudo first-order rate constants, k_{obs}, vs. the concentration of compound BB1.

FIG. 7A-E shows data on the inhibition of AEP in OGD-treated neurons. A. AEP activity, measured in primary neuronal cultures with 5 μM Cbz-Ala-Ala-Asn-AMC (x-axis denotes neurons were treated with 0.1 μM or 1.0 μM specified compound). B. Caspase activity, measured in primary neuronal cultures with 5 μM Ac-Asp-Glu-Val-Asp-AMC. C. Cathepsin activity, measured in primary neuronal cultures with 5 μM D-Val-Leu-Lys-AMC. D. Inhibition of APP cleavage. Lysates of primary cortical neurons pre-incubated with compounds for 30 min, underwent OGD for 4 hrs, were reperfused for 18 hrs (normoxia neurons remained at normoxic conditions). E. APP can be cleaved in a dose-dependent manner. Lysates of primary cortical neurons pre-incubated with compounds for 30 min, underwent OGD for 4 hrs, were reperfused for 18 hrs (normoxia neurons remained at normoxic conditions).

FIG. 8A-B shows data indicating compound 11 inhibits AEP activity and AEP-mediated cleavage of APP in the brain. A. AEP activity assay. 5XFAD mice were treated with compound 11 or vehicle at 10 mg/kg for 3 months. Compound 11 significantly decreased the activity of AEP in the brain. *P<0.01. B. Processing of APP by AEP and BACE1. Compound 11 decreased the AEP-generated APP fragment and BACE1-generated APP fragment (C99), but did not alter the level of full-length APP and BACE1.

FIG. 9A-G shows data indicating compound 11 alleviates Aβ deposition and cognitive impairment in 5XFAD mice. A. Thioflavin-S staining of amyloid plaques in the hippocampus (HP), motor cortex (MC), and frontal cortex (FC) of 5XFAD mouse brain sections. Scale bar, 50 μm. B. Quantitative analysis of amyloid plaques. The density of plaques in 5XFAD mouse brain was decreased by compound 11. *P<0.05, **P<0.01. C-D. Aβ1and Aβ1-42 ELISA in 5XFAD mice treated with compound 11 or vehicle. *P<0.05, **P<0.01 vs. vehicle-treated mice. E. Morris water maze analysis as distance traveled (mm) and integrated distance (AUC) for mice treated with compound 11 or vehicle. *P<0.05. F. The percentage of time spent in the target quadrant in the probe trail. Mice treated with compound 11 spend more time in the target quadrant than the vehicle-treated mice. **P<0.05. G. Swim speed of the 5XFAD mice were not altered by treatment with compound 11.

FIG. 10A-F shows data indicating compound 11 ameliorates synaptic loss and restores synaptic dysfunction in 5XFAD mice. A. Representative electron microscopy of the synaptic structures. Arrows indicate the synapses. B. Quantitative analysis of the synaptic density in vehicle—and compound 11-treated 5XFAD mice. Data are shown as mean ±SEM (n=3 mice per group). * p <0.01. C. Golgi staining reveals the dendritic spines from apical dendritic layer of the CA1 region. Scale bar, 5 μm. D. Quantitative analysis of the spine density. The decreased spine density in 5XFAD mice was reversed by compound 11. * P<0.01, ** P<0.05. E. Long-term potentiation (LTP) of fEPSPs was induced by 3× TBS (4 pulses at 100 Hz, repeated 3 times with a 200-ms interval). Shown traces are representative fEPSPs recorded at the time point 1 and 2 (vehicle treated), 3 and 4 (compound 11-treated mouse). The magnitude of LTP in 5XFAD mice was enhanced by treatment with compound 11. Data are presented as mean±SEM. *p<0.05 vs vehicle-treated mice. F. Synaptic transmission assessed by input/output (I/O) relation between stimuli intensity and fEPSP slope. I/O curves obtained in hippocampal slices prepared from vehicle- and compound 11-treated 5XFAD mice.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Terms

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

As used herein, the term “combination with” when used to describe administration with an additional treatment means that the agent may be administered prior to, together with, or after the additional treatment, or a combination thereof.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulfur atom or replacing an amino group with a hydroxy group. Contemplated derivative include switching carbocyclic, aromatic or phenyl rings with heterocyclic rings or switching heterocyclic rings with carbocyclic, aromatic or phenyl rings, typically of the same ring size. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze, all hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO₂Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)_{2O}Ra. Ra and Rb in this context may be the same or different and independently hydrogen, halogen hydroxy, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

As used herein, “alkyl” means a noncyclic straight chain or branched, unsaturated or saturated hydrocarbon such as those containing from 1 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3 -methyl- 1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkylthio” refers to an alkyl group as defined above attached through a sulfur bridge. An example of an alkylthio is methylthio, (i.e., —S—CH₃).

“Alkoxy” refers to an alkyl group as defined above attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above attached through an amino bridge. An example of an alkylamino is methylamino, (i.e., —NH—CH₃).

“Alkanoyl” refers to an alkyl as defined above attached through a carbonyl bridge (i.e., —(C═O)alkyl).

“Alkylsulfonyl” refers to an alkyl as defined above attached through a sulfonyl bridge (i.e., —S(═O)₂alkyl) such as mesyl and the like, and “Arylsulfonyl” refers to an aryl attached through a sulfonyl bridge (i.e., —S(═O)₂aryl).

“Alkylsulfamoyl” refers to an alkyl as defined above attached through a sulfamoyl bridge (i.e., —S(═O)₂NHalkyl), and an “Arylsulfamoyl” refers to an alkyl attached through a sulfamoyl bridge (i.e., —S(═O)₂NHaryl).

“Alkylsulfinyl” refers to an alkyl as defined above with the indicated number of carbon atoms attached through a sulfinyl bridge (i.e. —S(═O)alkyl).

The terms “halogen” and “halo” refer to fluorine, chlorine, bromine, and iodine.

The term “aroyl” refers to an aryl group (which may be optionally substituted as described above) linked to a carbonyl group (e.g., —C(O)-aryl).

The term “sulfamoyl” refers to the amide of sulfonic acid (i.e, —S(═O)₂NRR′)

An unspecified “R” group may be a hydrogen, lower alkyl, aryl, or heteroaryl, which may be optionally substituted with one or more, the same or different, substituents.

Throughout the specification, groups and substituents thereof may be chosen to provide stable moieties and compounds.

Inhibition of Asparagine Endopeptidase is Neuroprotective and Improves Cognitive Memory in a Mouse model of Alzheimer's Disease

Ageing is the greatest risk factor for Alzheimer's disease (AD). During ageing, the pH in brain gradually decreases. AEP is progressively upregulated in mouse brain and activated in aged mice. Moreover, AEP is also elevated and activated in human AD brains compared to normal controls. The active AEP cleaves both APP (amyloid precursor protein) and Tau, two major pathogenic players in AD. AEP processing APP facilitates BACE1 to degrade APP, leading to β-amyloid upregulation. Knockout of AEP from AD transgenic mouse models reverses the pathological events in 5XFAD and APP/PS1 mice and improves the cognitive deficit. On the other hand, active AEP proteolytically degrades tau, abolishes its microtubule assembly function, induces tau aggregation, and triggers neurodegeneration. Furthermore, AEP is activated in tau P301s is transgenic mice and human AD brain, leading to tau truncation in NFTs. Deletion of AEP from tau P301S transgenic mice substantially reduces NFTs deposit, alleviates the synapse loss and rescues impaired hippocampal synaptic plasticity and the cognitive deficits. AEP is primarily responsible for the hyperphosphorylation of tau through its cleavage of SET, a PP2A inhibitor after cleavage, which results in the inhibition of the enzyme responsible for 70% of tau phosphatase activity, Protein Phosphatase-2A (PP2A). See Basurto-Islas et al. AEP acts as a mediator in the onset and progression of AD. Inhibition of AEP can be a therapeutically useful for treating the neurodegenerative diseases including AD.

AEP is upregulated and activated in aged normal brain and human Alzheimer's Disease (AD) brain, playing a critical role in mediating the pathphysiology of AD. Disclosed herein are brain permeable and orally bioactive AEP inhibitor that reduces the senile plaque formation in AD mouse model and alleviates the cognitive defects. A high through-put screening was performed. Several skeletal families of compounds were discovered with potent inhibitory activities. A nontoxic and specific AEP inhibitor that was identified that selectively blocks AEP but not other related-cysteine proteases. Chronic treatment of 5XFAD mice with oral administration of the inhibitor ameliorates synapse loss and augments long-term potentiation (LTP), resulting in protection of memory loss in AD. Therefore, these findings indicate that these AEP inhibitors can be effective clinical therapeutic agents.

Stroke, seizures, and head trauma are all causative of brain tissue ischemia, which upregulates apoptotic and necrotic processes in brain tissue, implicating them as leading causes of neurodegeneration in humans. Depriving the brain of its blood supply induces an excitotoxic effect that causes neuronal death through an incompletely understood mechanism. A predominant feature of excitotoxicity is acidosis, which is a shift in the buffered brain interstitial pH from 7.3 to 6.0, resulting from increased cellular concentrations of the excitatory amino acid, glutamate. In response to the decrease in intracranial pH, caused by excitatory acidosis, AEP is activated and has been shown to display aberrant activity toward one of its substrates, SET, a DNAse inhibitor. SET is a phosphoprotein and is predominantly localized to the nucleus, where it is involved in transcriptional regulation through interactions with histone tails. SET also acts as a mediator of apoptosis, by inhibiting DNA nicking, in the Granzyme-A-mediated cell death pathway. AEP is activated following induction of ischemia and acidosis, and proteolytically cleaves SET, which results in neuronal cell death; whereas, SET remains intact in AEP-deficient mice and neuronal cell death is negligible. This observation suggests that AEP inhibition provides a way to prevent neurodegeneration following stroke, seizure or head trauma.

AEP is primarily responsible for the hyperphosphorylation of tau through its cleavage of SET, which results in the inhibition of the enzyme responsible for 70% of tau phosphatase activity, Protein Phosphatase-2A (PP2A). The levels of active AEP and cleaved N-terminal and C-terminal fragments of SET are elevated in the brains of AD patients; additionally, acidosis was found to trigger the cytoplasmic translocation of AEP and SET from the lysosome and nucleus, respectively. This finding indicates that AEP seems to play a role in the etiopathogenesis of Alzheimer's Disease.

AD is the most common neurodegenerative diseases. It is characterized by the deposition of Aβ and insoluble tau. We found that AEP cleaves APP and tau in the AD brain. Compared to the full-length APP, the AEP-generated APP fragment is a better substrate for β-secretase, thus enhance the production of Aβ. Tau cleavage by AEP will generate several fragments that can promote it deposition. Furthermore, cleavage of SET by AEP promotes neuronal death induced by ischemia, and promotes the phosphorylation of tau. All these observations indicate AEP inhibitors may rescue the progressive neurodegeneration in AD. To test this hypothesis, 5XFAD mice were treated with compound 11 for 3 months and monitored its effect on the morphological, electrophysiological, biochemical and behavioral changes in this AD model. Chronic administration of compound 11 decreased the deposition of Aβ, presumably by inhibiting the cleavage of APP by AEP. The restoration of cognitive function by compound 11 indicates the protective effect of AEP inhibitor in vitro may translate into clinical benefit. Synaptic dysfunction is the early and invariant feature of AD. Synaptic loss correlates to dementia in AD. 5XFAD mice show decreased synaptic density and LTP magnitude compared to the non-transgenic mice. Compound 11 reversed the synaptic dysfunction as shown by increased synaptic density and LTP magnitude, indicating an “synaptoprotective” effect of the compound.

### Asparagine Endopeptidase Inhibitors

This disclosure relates to asparagine endopeptidase inhibitors. In some embodiments, the asparagine endopeptidase inhibitor is a substituted benzo[c][1,2,5] oxadiazole derivative such as a compound of the following formula:

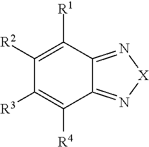

prodrugs, esters, derivatives, or salts thereof wherein,

X is O or S;

R¹ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R¹ is optionally substituted with one or more, the same or different, R¹⁰;

R¹⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R¹⁰ is optionally substituted with one or more, the same or different, R¹¹;

R¹¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R² is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R² is optionally substituted with one or more, the same or different, R²⁰;

R²⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R²⁰ is optionally substituted with one or more, the same or different, R²¹;

R²¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R³ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R³ is optionally substituted with one or more, the same or different, R³⁰;

R³⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R³⁰ is optionally substituted with one or more, the same or different, R³¹;

R³¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R⁴ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁴ is optionally substituted with one or more, the same or different, R⁴⁰;

R⁴⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁴⁰ is optionally substituted with one or more, the same or different, R⁴¹; and

R⁴¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

In certain embodiments, R¹ is amino. In certain embodiments, R² is hydrogen. In certain embodiments, R³ is hydrogen. In certain embodiments, R⁴ is heterocycyl.

In some embodiments, the asparagine endopeptidase inhibitor is a substituted 3,7-dihydropurine-2,6-dione derivative such as a compound of the following formula:

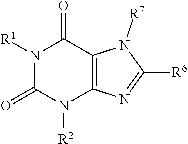

prodrugs, esters, derivatives, or salts thereof wherein,

R¹ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R¹ is optionally substituted with one or more, the same or different, R¹⁰;

R¹⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R¹⁰ is optionally substituted with one or more, the same or different, R¹¹;

R¹¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R² is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R² is optionally substituted with one or more, the same or different, R²⁰;

R²⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R²⁰ is optionally substituted with one or more, the same or different, R²¹;

R²¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R⁶ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁶ is optionally substituted with one or more, the same or different, R⁶⁰;

R⁶⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁶⁰ is optionally substituted with one or more, the same or different, R⁶¹;

R⁶¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R⁷ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁷ is optionally substituted with one or more, the same or different, R⁷⁰;

R⁷⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁷⁰ is optionally substituted with one or more, the same or different, R⁷¹; and

R⁷¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

In certain embodiments, R¹ is alkyl.

In certain embodiments, R² is alkyl.

In certain embodiments, R⁶ is mercapto.

In certain embodiments, R⁷ is alkyl.

In some embodiments, the asparagine endopeptidase inhibitor is a substituted 1,3,4-thiadiazole derivative such as a compound of the following formula:

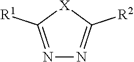

prodrugs, esters, derivatives, or salts thereof wherein,

X is O or s;

R¹ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R¹ is optionally substituted with one or more, the same or different, R¹⁰;

R¹⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R¹⁰ is optionally substituted with one or more, the same or different, R¹¹;

R¹¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R² is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R² is optionally substituted with one or more, the same or different, R²⁰;

R²⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R²⁰ is optionally substituted with one or more, the same or different, R²¹; and

R²¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

In certain embodiments, R¹ is mercapto. In certain embodiments, R² is amino.

In some embodiments, the asparagine endopeptidase inhibitor is a substituted 1-phenyl-1H-pyrrole-2,5-dione derivative such as a compound of the following formula:

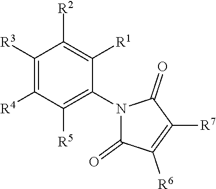

prodrugs, esters, derivatives, or salts thereof wherein,

R¹ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R¹ is optionally substituted with one or more, the same or different, R¹⁰;

R¹⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R¹⁰ is optionally substituted with one or more, the same or different, R¹¹;

R¹¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R² is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R² is optionally substituted with one or more, the same or different, R²⁰;

R²⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R²⁰ is optionally substituted with one or more, the same or different, R²¹;

R²¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R³ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R³ is optionally substituted with one or more, the same or different, R³⁰;

R³⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R³⁰ is optionally substituted with one or more, the same or different, R³¹;

R³¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R⁴ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁴ is optionally substituted with one or more, the same or different, R⁴⁰;

R⁴⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁴⁰ is optionally substituted with one or more, the same or different, R⁴¹;

R⁴¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R⁵ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁵ is optionally substituted with one or more, the same or different, R⁵⁰;

R⁵⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁵⁰ is optionally substituted with one or more, the same or different, R⁵¹;

R⁵¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R⁶ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁶ is optionally substituted with one or more, the same or different, R⁶⁰;

R⁶⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁶⁰ is optionally substituted with one or more, the same or different, R⁶¹;

R⁶¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R⁷ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁷ is optionally substituted with one or more, the same or different, R⁷⁰;

R⁷⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁷⁰ is optionally substituted with one or more, the same or different, R⁷¹; and

R⁷¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

In some embodiments, the asparagine endopeptidase inhibitor is a substituted 1-methylpiperazine derivative such as a compound of the following formula:

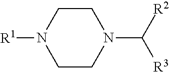

prodrugs, esters, derivatives, or salts thereof wherein,

R¹ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R¹ is optionally substituted with one or more, the same or different, R¹⁰;

R¹⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R¹⁰ is optionally substituted with one or more, the same or different, R¹¹;

R¹¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R² is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R² is optionally substituted with one or more, the same or different, R²⁰;

R²⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R²⁰ is optionally substituted with one or more, the same or different, R²¹;

R²¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R³ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R³ is optionally substituted with one or more, the same or different, R³⁰;

R³⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R³⁰ is optionally substituted with one or more, the same or different, R³¹; and

R³¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

In some embodiments, the asparagine endopeptidase inhibitor is a substituted quinolin-5-ylmethanamine derivative such as a compound of the following formula:

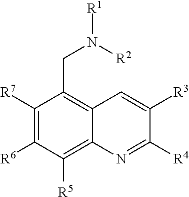

prodrugs, esters, derivatives, or salts thereof wherein,

R¹ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R¹ is optionally substituted with one or more, the same or different, R¹⁰;

R¹⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R¹⁰ is optionally substituted with one or more, the same or different, R¹¹;

R¹¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R² is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R² is optionally substituted with one or more, the same or different, R²⁰;

R²⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R²⁰ is optionally substituted with one or more, the same or different, R²¹;

R²¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R³ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R³ is optionally substituted with one or more, the same or different, R³⁰;

R³⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R³⁰ is optionally substituted with one or more, the same or different, R³¹;

R³¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R⁴ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁴ is optionally substituted with one or more, the same or different, R⁴⁰;

R⁴⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁴⁰ is optionally substituted with one or more, the same or different, R⁴¹;

R⁴¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R⁵ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁵ is optionally substituted with one or more, the same or different, R⁵⁰;

R⁵⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁵⁰ is optionally substituted with one or more, the same or different, R⁵¹;

R⁵¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R⁶ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁶ is optionally substituted with one or more, the same or different, R⁶⁰;

R⁶⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁶⁰ is optionally substituted with one or more, the same or different, R⁶¹;

R⁶¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R⁷ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁷ is optionally substituted with one or more, the same or different, R⁷⁰;

R⁷⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁷⁰ is optionally substituted with one or more, the same or different, R⁷¹; and

R⁷¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

In some embodiments, the asparagine endopeptidase inhibitor is a substituted thiazole derivative such as a compound of the following formula:

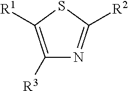

prodrugs, esters, derivatives, or salts thereof wherein,

R¹ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R¹ is optionally substituted with one or more, the same or different, R¹⁰;

R¹⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R¹⁰ is optionally substituted with one or more, the same or different, R¹¹;

R¹¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R² is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R² is optionally substituted with one or more, the same or different, R²⁰;

R²⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R²⁰ is optionally substituted with one or more, the same or different, R²¹;

R²¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R³ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R³ is optionally substituted with one or more, the same or different, R³⁰;

R³⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R³⁰ is optionally substituted with one or more, the same or different, R³¹; and

R³¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

In some embodiments, the asparagine endopeptidase inhibitor is a substituted 6-methyl-5,6,7,8-tetrahydropyrido[4,3-d]pyrimidin-4(3H)-one derivative such as a compound of the following formula:

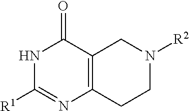

prodrugs, esters, derivatives, or salts thereof wherein,

R¹ is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R¹ is optionally substituted with one or more, the same or different, R¹⁰;

R¹⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R¹⁰ is optionally substituted with one or more, the same or different, R¹¹;

R¹¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl;

R² is selected from hydrogen, alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R² is optionally substituted with one or more, the same or different, R²⁰;

R²⁰ is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R²⁰ is optionally substituted with one or more, the same or different, R²¹; and

R²¹ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

### Pharmaceutical Compositions

In certain embodiments, the disclosure relates to pharmaceutical compositions comprising a compound disclosed herein and a pharmaceutically acceptable excipient. In certain embodiments, the pharmaceutical composition is in the form of a pill, capsule, tablet, or saline aqueous buffer.

In certain embodiments, the pharmaceutically acceptable excipient is selected from a saccharide, disaccharide, sucrose, lactose, glucose, mannitol, sorbitol, polysaccharides, starch, cellulose, microcrystalline cellulose, cellulose ether, hydroxypropyl cellulose (HPC), xylitol, sorbitol, maltito, gelatin, polyvinylpyrrolidone (PVP), polyethylene glycol (PEG), hydroxypropyl methylcellulose (HPMC), crosslinked sodium carboxymethyl cellulose, dibasic calcium phosphate, calcium carbonate, stearic acid, magnesium stearate, talc, magnesium carbonate, silica, vitamin A, vitamin E, vitamin C, retinyl palmitate, selenium, cysteine, methionine, citric acid, and sodium citrate, methyl paraben, propyl paraben, and combinations thereof.

Pharmaceutical compositions disclosed herein may be in the form of pharmaceutically acceptable salts, as generally described below. Some preferred, but non-limiting examples of suitable pharmaceutically acceptable organic and/or inorganic acids are hydrochloric acid, hydrobromic acid, sulfuric acid, nitric acid, acetic acid and citric acid, as well as other pharmaceutically acceptable acids known per se (for which reference is made to the references referred to below).

When the compounds of the disclosure contain an acidic group as well as a basic group, the compounds of the disclosure may also form internal salts, and such compounds are within the scope of the disclosure. When the compounds of the disclosure contain a hydrogen-donating heteroatom (e.g. NH), the disclosure covers salts and/or isomers formed by transfer of said hydrogen atom to a basic group or atom within the molecule.

Pharmaceutically acceptable salts of the compounds include the acid addition and base salts thereof Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds described herein may be administered in the form of prodrugs. A prodrug may include a covalently bonded carrier which releases the active parent drug when administered to a mammalian subject. Prodrugs may be prepared by modifying functional groups present in the compounds in such a way that the modifications are cleaved, either in routine manipulation or in vivo, to the parent compounds. Prodrugs include, for example, compounds wherein a hydroxy group is bonded to any group that, when administered to a mammalian subject, cleaves to form a free hydroxy group. Examples of prodrugs include, but are not limited to, acetate, formate and benzoate derivatives of alcohol functional groups in the compounds. Methods of structuring a compound as prodrugs may be found in the book of Testa and Mayer, Hydrolysis in Drug and Prodrug Metabolism, Wiley (2006). Typical prodrugs form the active metabolite by transformation of the prodrug by hydrolytic enzymes, the hydrolysis of amide, lactams, peptides, carboxylic acid esters, epoxides or the cleavage of esters of inorganic acids. It is well within the ordinary skill of the art to make an ester prodrug, e.g., acetyl ester of a free hydroxy group. It is well known that ester prodrugs are readily degraded in the body to release the corresponding alcohol. See e.g., Imai, Drug Metab Pharmacokinet. (2006) 21(3):173-85, entitled “Human carboxylesterase isozymes: catalytic properties and rational drug design.”

Pharmaceutical compositions for use in the present disclosure typically comprise an effective amount of a compound and a suitable pharmaceutical acceptable carrier. The preparations may be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is made to U.S. Pat. No. 6,372,778, U.S. Pat. No. 6,369,086, U.S. Pat. No. 6,369,087 and U.S. Pat. No. 6,372,733 and the further references mentioned above, as well as to the standard handbooks, such as the latest edition of Remington's Pharmaceutical Sciences.

Generally, for pharmaceutical use, the compounds may be formulated as a pharmaceutical preparation comprising at least one compound and at least one pharmaceutically acceptable carrier, diluent or excipient and/or adjuvant, and optionally one or more further pharmaceutically active compounds.

The pharmaceutical preparations of the disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the disclosure, e.g. about 10, 25, 50, 100, 200, 300 or 400 mg per unit dosage.

The compounds may be administered by a variety of routes including the oral, ocular, rectal, transdermal, subcutaneous, intravenous, intramuscular or intranasal routes, depending mainly on the specific preparation used. The compound will generally be administered in an “effective amount”, by which is meant any amount of a compound that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the patient per day, more often between 0.1 and 500 mg, such as between 1 and 250 mg, for example about 5, 10, 20, 50, 100, 150, 200 or 250 mg, per kilogram body weight of the patient per day, which may be administered as a single daily dose, divided over one or more daily doses. The amount(s) to be administered, the route of administration and the further treatment regimen may be determined by the treating clinician, depending on factors such as the age, gender and general condition of the patient and the nature and severity of the disease/symptoms to be treated. Reference is made to U.S. Pat. No. 6,372,778, U.S. Pat. No. 6,369,086, U.S. Pat. No. 6,369,087 and U.S. Pat. No. 6,372,733 and the further references mentioned above, as well as to the standard handbooks, such as the latest edition of Remington's Pharmaceutical Sciences.

For an oral administration form, the compound may be mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation may be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, the compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the disclosure or their physiologically tolerable salts in a pharmaceutically acceptable solvent, such as ethanol or water, or a mixture of such solvents. If required, the formulation may contain other pharmaceutical auxiliaries such as surfactants, emulsifiers and stabilizers as well as a propellant.

For subcutaneous or intravenous administration, the compounds, if desired with the substances customary therefore such as solubilizers, emulsifiers or further auxiliaries are brought into solution, suspension, or emulsion. The compounds may also be lyophilized and the lyophilizates obtained used, for example, for the production of injection or infusion preparations. Suitable solvents are, for example, water, physiological saline solution or alcohols, e.g. ethanol, propanol, glycerol, sugar solutions such as glucose or mannitol solutions, or mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, the formulations may be prepared by mixing the compounds with a suitable non-irritating excipient, such as cocoa butter, synthetic glyceride esters or polyethylene glycols, which are solid at ordinary temperatures, but liquefy and/or dissolve in the rectal cavity to release the drug.

In certain embodiments, it is contemplated that these compositions may be extended release formulations. Typical extended release formations utilize an enteric coating. Typically, a barrier is applied to oral medication that controls the location in the digestive system where it is absorbed. Enteric coatings prevent release of medication before it reaches the small intestine. Enteric coatings may contain polymers of polysaccharides, such as maltodextrin, xanthan, scleroglucan dextran, starch, alginates, pullulan, hyaloronic acid, chitin, chitosan and the like; other natural polymers, such as proteins (albumin, gelatin etc.), poly-L-lysine; sodium poly(acrylic acid); poly(hydroxyalkylmethacrylates) (for example poly(hydroxyethyl methacrylate)); carboxypolymethylene (for example Carbopol™); carbomer; polyvinylpyrrolidone; gums, such as guar gum, gum arabic, gum karaya, gum ghatti, locust bean gum, tamarind gum, gellan gum, gum tragacanth, agar, pectin, gluten and the like; poly(vinyl alcohol); ethylene vinyl alcohol; polyethylene glycol (PEG); and cellulose ethers, such as hydroxymethylcellulose (HMC), hydroxyethylcellulose (HEC), hydroxypropylcellulose (HPC), methylcellulose (MC), ethylcellulose (EC), carboxyethylcellulose (CEC), ethylhydroxyethylcellulose (EHEC), carboxymethylhydroxyethylcellulose (CMHEC), hydroxypropylmethyl-cellulose (HPMC), hydroxypropylethylcellulose (HPEC) and sodium carboxymethylcellulose (Na CMC); as well as copolymers and/or (simple) mixtures of any of the above polymers. Certain of the above-mentioned polymers may further be crosslinked by way of standard techniques.

The choice of polymer will be determined by the nature of the active ingredient/drug that is employed in the composition of the invention as well as the desired rate of release. In particular, it will be appreciated by the skilled person, for example in the case of HPMC, that a higher molecular weight will, in general, provide a slower rate of release of drug from the composition. Furthermore, in the case of HPMC, different degrees of substitution of methoxyl groups and hydroxypropoxyl groups will give rise to changes in the rate of release of drug from the composition. In this respect, and as stated above, it may be desirable to provide compositions of the invention in the form of coatings in which the polymer carrier is provided by way of a blend of two or more polymers of, for example, different molecular weights in order to produce a particular required or desired release profile.

Microspheres of polylactide, polyglycolide, and their copolymers poly(lactide-co-glycolide) may be used to form sustained-release protein or compound delivery systems. Proteins and/or compounds may be entrapped in the poly(lactide-co-glycolide) microsphere depot by a number of methods, including formation of a water-in-oil emulsion with water-borne protein and organic solvent-borne polymer (emulsion method), formation of a solid-in-oil suspension with solid protein dispersed in a solvent-based polymer solution (suspension method), or by dissolving the protein in a solvent-based polymer solution (dissolution method). One may attach poly(ethylene glycol) to proteins (PEGylation) to increase the in vivo half-life of circulating therapeutic proteins and decrease the chance of an immune response.

### Methods of Use

In certain embodiments, the asparagine endopeptidase inhibitors are useful for treating or preventing neurodegenerative diseases and cognitive disorders such as Alzheimer's Disease. In certain embodiments, the disclosure relates to pharmaceutical compositions comprising an asparagine endopeptidase inhibitor and a pharmaceutically acceptable excipient. In certain embodiments, the disclosure relates to methods of treating or preventing a neurodegenerative disease comprising administering an effective amount of pharmaceutical composition a asparagine endopeptidase inhibitor disclosed herein to a subject in need thereof.

In certain embodiments, the subject is at risk of or exhibiting symptoms of AD.

In certain embodiments, the disclosure contemplates administering compounds disclosed herein in combination with an imaging agent such as florbetapir (¹⁸F) and/or a therapeutic agent related to treating or ameliorating one or more symptoms of AD.

In certain embodiments, the disclosure contemplates administering compounds disclosed herein in combination with medications for memory loss, treatments for behavioral changes, treatments for sleep changes.

In certain embodiments, the disclosure contemplates administering compounds disclosed herein in combination with medication selected from cholinesterase inhibitors such as donepezil, rivastigmine, galantamine, and tacrine and/or an agent for blocking NMDA receptor such as memantine to treat the cognitive symptoms (memory loss, confusion, and problems with thinking and reasoning) of Alzheimer's disease.

In certain embodiments, the disclosure contemplates administering compounds disclosed herein in combination with Vitamin E.

In certain embodiments, the disclosure contemplates administering compounds disclosed herein in combination with medications such as anti-irritability, anti-anxiety, anti-psychotic, anti-insomnia, and anti-depression agents.

In certain embodiments, the disclosure contemplates administering compounds disclosed herein in combination with monoclonal antibody vaccines to amyloid including but not limited to solanuzemab, gantenerumab, and bapineuzumab.

In certain embodiments, the disclosure contemplates administering compounds disclosed herein in combination with medications for stroke or traumatic brain injury.

In certain embodiments, the disclosure contemplates administering compounds disclosed herein in combination with with recombinant tissue plasminogen activator (rtPA).

In certain embodiments, compounds disclosed herien can be used to treat a variety of diseases associated with apoptosis including neurodegenerative disorders, ischemic injuries, acquired immunodeficiency syndrome (AIDS), and osteoporosis. Apoptosis is involved in amyotrophic lateral sclerosis (ALS), Huntington's disease, Alzheimer's disease, Parkinson's disease, and spinal muscular atrophy. In multiple sclerosis (MS), the death of the oligodendrocytes is an important example of the glial degeneration through apoptosis.

In certain embodiments, compounds disclosed herein would be useful for the treatment of Huntington's disease and other neurodegenerative diseases such as dentatorubropallidoluysian atrophy (DRPLA), spinocerebellar atrophy type 3 (SCA-3), and spinal bulbar muscular atrophy (SBMA).

Neuronal apoptosis is also seen after acute injuries such as stroke, trauma, and ischemia. Apoptosis has been observed in striatal and cortical neurons in animal models of stroke.

### EXPERIMENTAL

Inhibitors of AEP were Identified by High-Throughput Screen

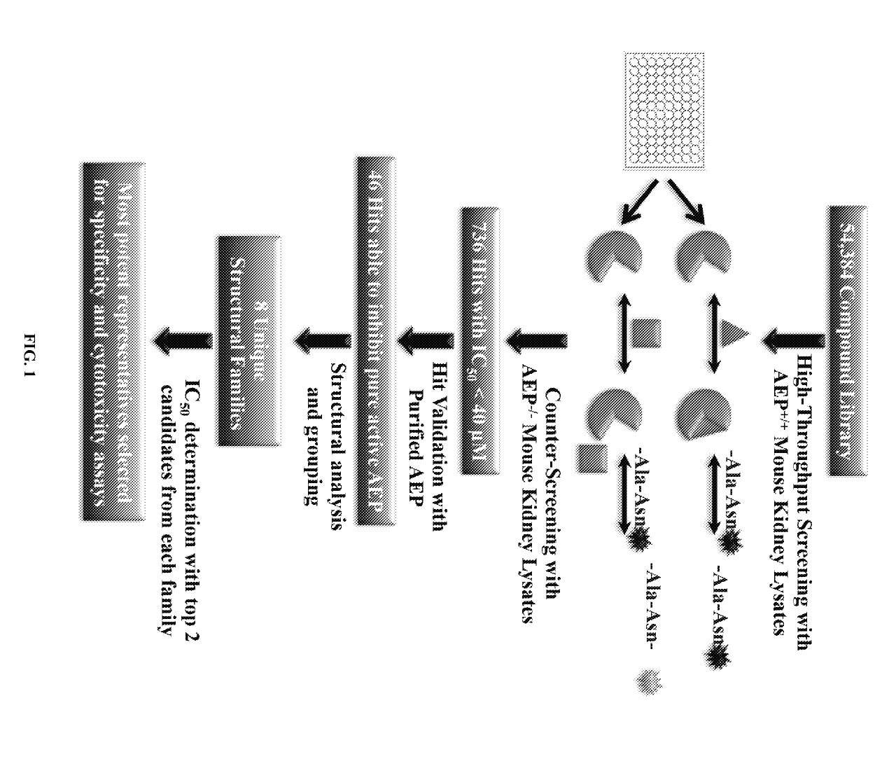

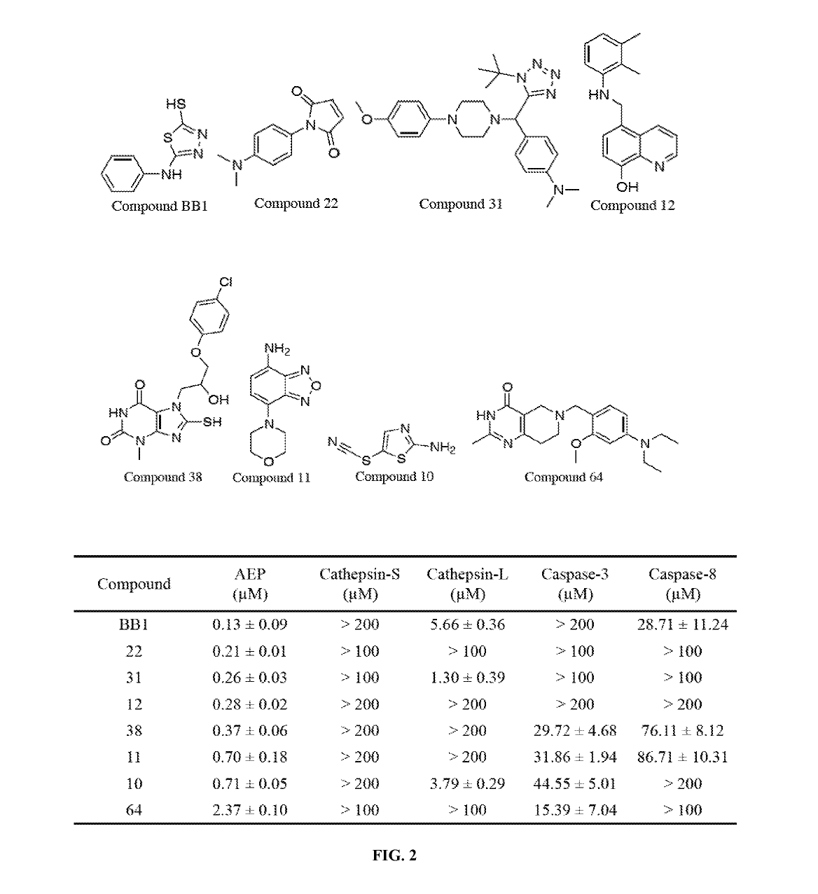

To identify small-molecule inhibitors of AEP, a high-throughput screen was designed in conjunction with the Emory Chemical Biology Discovery Center. The screen incorporated mouse kidney lysates to assay a 54,384 compound library. Upon counter-screening with kidney lysates from AEP−/− mice, 736 hits were confirmed to display IC₅₀ values toward the cellular AEP less than or equal to 40 μM. A third screen with purified active AEP found that 46 hits exhibited promising inhibitory activity (FIG. 1). Additional structural analysis and grouping allowed the compounds to be sorted into 8 distinct backbone families. After some of the most potent compounds from each group were tested with purified active AEP, IC₅₀ values for the top 8 candidates were found. The specificity of the compounds was also determined using four major cysteine proteases (FIG. 2). Compound BB1 appeared to possess the greatest potency toward AEP, at about 130 nM, and it was about 38-fold more selective for AEP than Cathepsin-L. Compound 22 on the other hand, had an IC₅₀ greater than 100 μM for all of the tested cysteine proteases. Compounds 11 and 38 also seemed to be potent inhibitors of AEP, since they displayed IC₅₀ values of approximately 700 and 370 nM, respectively, and they were at least 80- fold more selective for AEP than Caspase-3 or Caspase-8. Compound 64 exhibited the highest IC₅₀ at 2.37 μM. It is a low-micromolar inhibitor and it is over 40-fold more selective for AEP as compared to Cathepsin-S, Cathepsin-L and Caspase-8, and about 6-fold more selective for Caspase-3.

### Cell Permeability of the Compounds

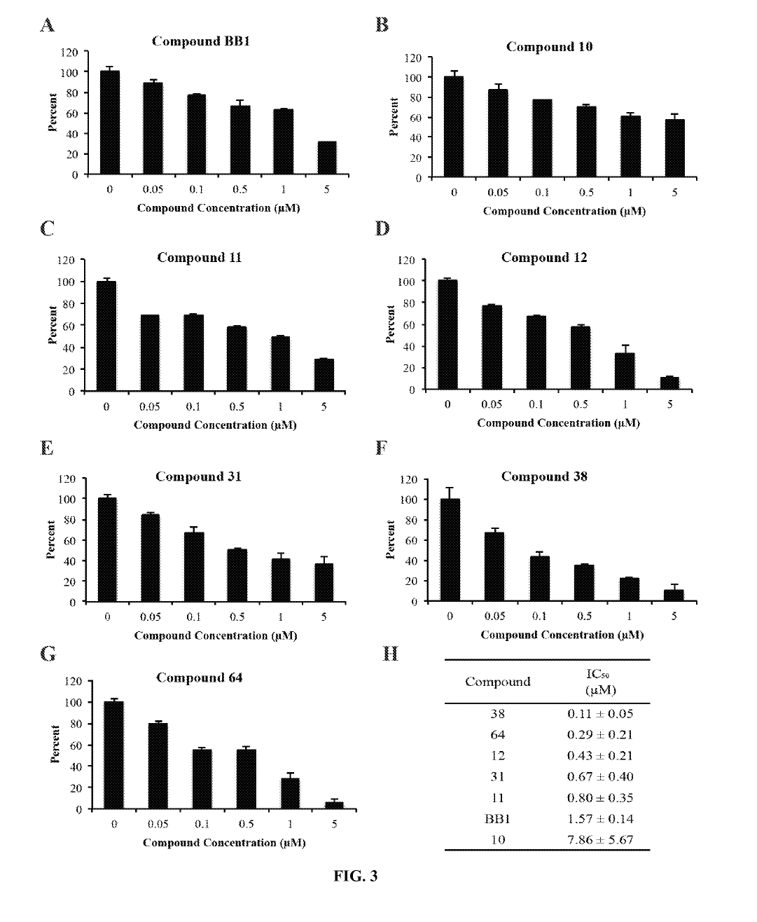

In an attempt to assess the activity of the compounds in intact cells, the compounds were incubated with human B lymphoblastoid pala cells, which are rich in endogenous AEP and have been used for inhibiting AEP in cellular assay. Most of the compounds were able to inhibit the enzyme with IC₅₀ values in the sub-micromolar range, however compounds BB1 and 10 exhibited slightly larger IC₅₀ values; many of the compounds seem to be cell permeable (FIG. 3). ADME characteristics of compounds with IC₅₀ values<1 μM were further evaluated.

### In Vitro ADMET Profiles

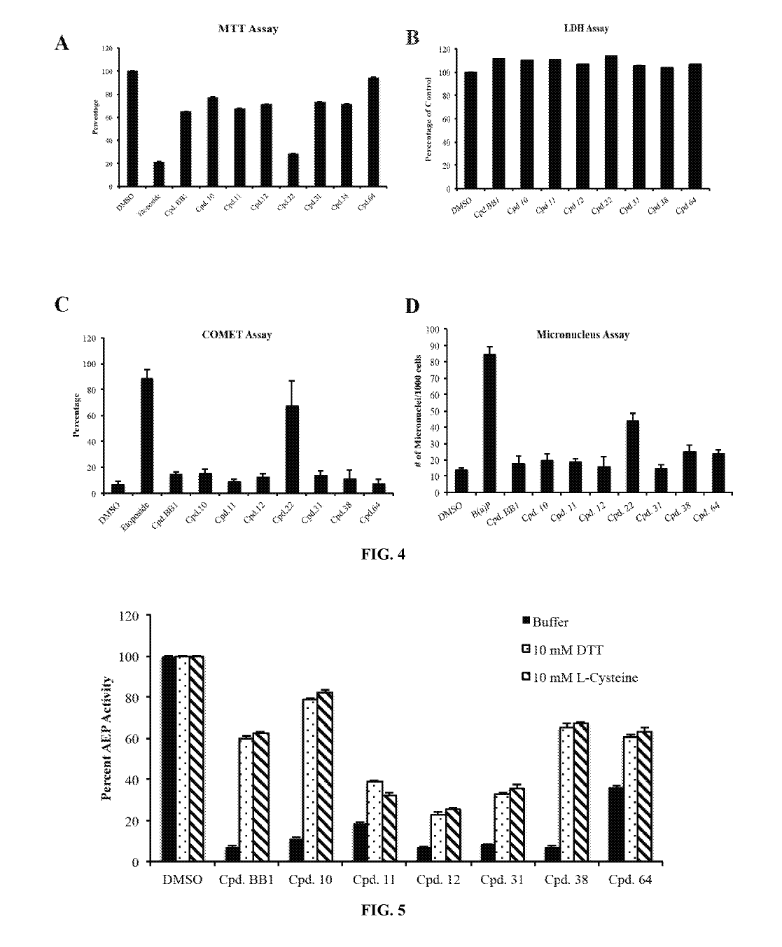

In an effort to characterize the toxicity of the compounds, an MTT assay was performed using human hepatocellular carcinoma, HepG2 cells and primary culture neurons to monitor the cell viability. In HepG2 cells, compound 22, the maleimide-containing derivative, revealed a similar toxicity to the positive control etoposide, which is a topoisomerase inhibitor and known to induce double strand breaks (FIG. 4A). The cytotoxicity of the compounds in primary neuronal cultures was determined using an LDH assay to measure cytolysis (FIG. 4B). The carcinogenicity of a compound is directly proportional to its induction of micronuclei. To assess whether compounds possess any carcinogenicity, a COMET assay was performed and a micronucleus assay. Benzo(α)pyrene (B(a)p) that generates measurable DNA nicks within these assays was included as a positive control. Following treatment for 24 hr with 50 μM compound, compound 22 was the only test compound to show genotoxicity, thus, it was excluded from further analyses (FIGS. 4C and 4D).

To explore the in vitro ADME profiles of the compounds, additional tests were conducted. A Caco-2 monolayer permeability screen was performed to assess the absorption characteristics of the compounds and compounds 11 and 31 were found to be highly permeable and should therefore be well physiologically absorbed (Table 1).

TABLE 1
Caco-2 permeability.

| | A -> B Pₐₚₚ | B -> A Pₐₚₚ | |
|---|---|---|---|
| Compound | (10⁻⁶ cm · s⁻¹) | (10⁻⁶ cm · s⁻¹) | R_{E} |
| Ranitidine | 0.8 | 2.5 | 3.2 |
| Warfarin | 28.5 | 12.6 | 0.4 |
| Talinolol | 0.3 | 6.0 | 23.9 |
| 11 | 35.0 | 7.0 | 0.2 |
| 12 | 1.4 | 3.7 | 2.7 |
| 31 | 19.3 | 17.4 | 0.9 |
| 38 | 1.1 | 20.3 | 18.2 |
| 64 | <LLOQ | <LLOQ | NA |

LLOQ = Compound not detected on receiver side

In the BBB-PAMPA permeability assay, compound 11 was detected at high levels and was thus considered able to cross the blood-brain barrier (Table 2).

TABLE 2
BBB-PAMPA permeability.

| | Pₑ |
|---|---|
| Compound | (10⁻⁶ cm · s⁻¹) |
| Theophylline | 0.12 |
| Verapamil | 17.2 |
| 11 | >25 |
| 12 | <LLOQ |
| 31 | <LLOQ |
| 38 | 0.007 |
| 64 | ND |

LLOQ = Compound not detected on receiver side
ND = Peak not detected due to bioanalysis issue

The human liver microsomal stability screen demonstrated that following 30 min of incubation, 76% of compound 11 and 88% of compound 38 remained in human liver microsomes (Table 3).

TABLE 3
Liver microsomal stability.

| | | Mean Remaining | Mean Remaining |
|---|---|---|---|
| | | Parent | Parent |
| Compound | Species | (with NADPH) | (NADPH-free) |
| Verapamil | Human | 4.6% | 101% |
| | Mouse | 2.6% | 101% |
| Warfarin | Human | 96% | 104% |
| | Mouse | 91% | 100% |
| 11 | Human | 76% | 93% |
| | Mouse | 20% | 103% |
| 12 | Human | 1% | 1% |
| | Mouse | 1% | 0% |
| 31 | Human | 12% | 75% |
| | Mouse | 16% | 86% |
| 38 | Human | 88% | 107% |
| | Mouse | 98% | 99% |
| 64 | Human | ND* | ND* |
| | Mouse | ND* | ND* |

ND = Peak not detected due to bioanalysis issue (poor ionization)

According to CYP inhibition screening, compound 31 was able to inhibit CYP2C9 at 69.2% and CYP2C19 at 55.7%, while compound 64 inhibited CYP2D6 at 58.3% at 10 μM of concentration, suggesting that these two compounds can be capable of producing potential drug-drug interactions (Table 4).

TABLE 4
CYP inhibition.

| | Test | | | | | | |
|---|---|---|---|---|---|---|---|
| | Concentration | CYP3A4- | CYP3A- | | | | |
| Compound | (μM) | Midazolam | Testosterone | CYP2C9 | CYP2D6 | CYP2C19 | CYP1A2 |
| 11 | 10 | 8.0% | 21.6% | 5.6% | 21.9% | 6.8% | 37.0% |
| | 3 | 3.6% | 4.1% | 4.3% | 7.8% | 4.4% | 13.1% |
| 12 | 10 | −2.5% | 27.6% | −2.7% | −2.6% | 29.4% | 44.5% |
| | 3 | −2.5% | 14.2% | 4.3% | 2.9% | 13.4% | 28.4% |
| 31 | 10 | 18.1% | 0.7% | 69.2% | 1.4% | 55.7% | 10.3% |
| | 3 | −0.9% | 2.5% | 47.8% | 9.9% | 37.7% | 13.5% |
| 38 | 10 | 10.4% | 2.4% | 17.1% | 0.9% | 9.5% | 10.5% |
| | 3 | 7.3% | −6.0% | 15.6% | −5.4% | −12.0% | −7.6% |
| 64 | 10 | 10.1% | −0.1% | 7.9% | 58.3% | 20.4% | 22.2% |
| | 3 | 0.6% | 1.5% | 4.6% | 34.6% | 31.6% | 23.7% |

### DTT Reversibility of the Compounds

Although it is not intended that embodiments of this disclosure be limited by any particular mechanism, to gain additional insight into the mechanism utilized by the inhibitors for abrogation of AEP activity, their reversibility in the presence of free thiols was determined. The addition of a strong reducing agent, such as DTT, or a weaker reducing agent, L-cysteine, to an inhibited reaction can be used to out-compete the inhibitory agent and restore catalytic activity to an enzyme with an active-site thiol residue. Here, a similar approach was used by adding a reducing agent, either DTT or L-cysteine, to a reaction, in which AEP had been incubated with a specified inhibitor, in an attempt to reverse the effects of the inhibitor. The thiol-containing compounds, BB1 and 38, and compound 10, which contains a thiocyanate moiety, all appeared to have regained a substantial amount of activity in the presence of the reducing agents, indicating that the reducing agents were able to reduce the active-site cysteine of the enzyme and thus increase the effective concentration of active enzyme. However, in the presence of the sulfur-containing compounds, BB1, 10 and 38, AEP regained a substantial amount of activity. For compounds BB1 and 38, this increase in enzymatic activity may be due to the reduction of a disulfide linkage between the inhibitor and the enzyme, since these compounds contain thiols. Compound 10 contains a thiocyanate and may undergo nucleophilic attack by the enzyme's active-site thiolate to form a thioimidate enzyme-inhibitor complex. Under acidic conditions, this complex is reducible by either a strong reducing agent, such as DTT, or the weaker L-cysteine. Thus, it is possible that compounds BB1, 10 and 38 may form covalent bonds with the active-site cysteine of AEP and competitively inhibit its activity.

### Inhibitor Characterization

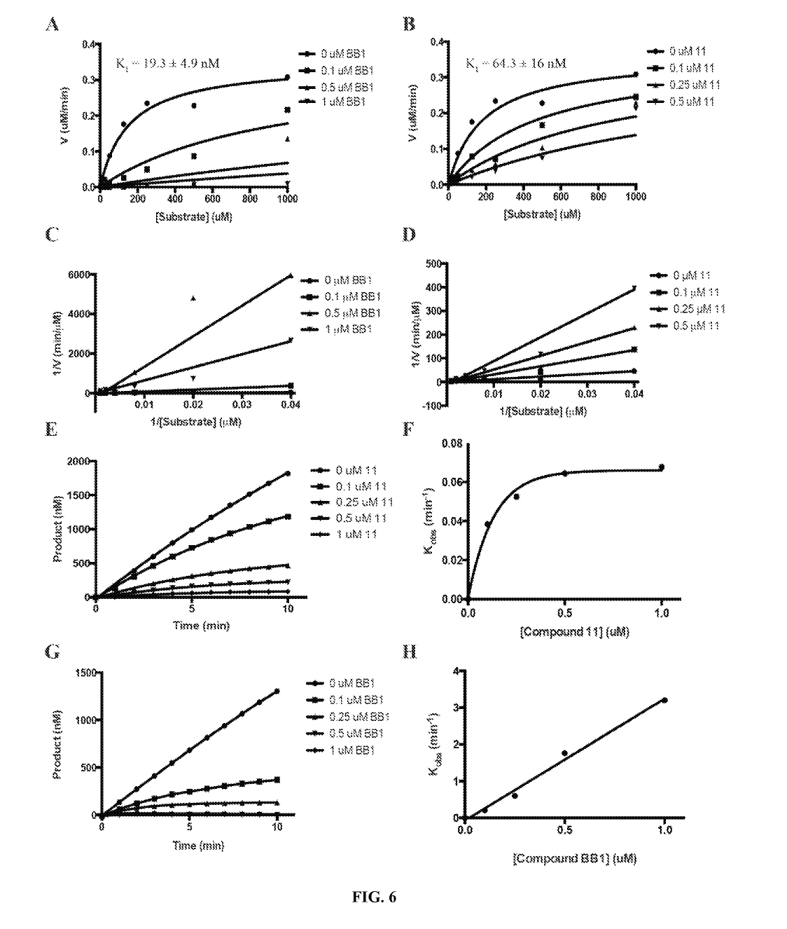

To continue to assess the mechanism utilized by the compounds to inhibit AEP, the inhibition kinetics were determined for compound, 11, as well as compound BB1, in an attempt to confirm that the thiol moiety is competitively inhibiting the enzyme. In order to determine the mode of inhibition of compounds BB1 and 11, steady-state kinetic parameters were measured in the presence of increasing concentrations of each inhibitor (FIG. 6A-B). The resulting Michaelis-Menten plots for each compound seem be indicative of competitive inhibition. Competitive inhibitors compete with substrate for binding at the active site of the enzyme, thus at saturating substrate concentrations, inhibition can be attenuated. The inhibitor constant, K_{I} is the concentration of inhibitor that produces half-maximal inhibition and is a measurement of an inhibitor's potency; the K_{I} values for the compounds are listed in FIG. 6A-B. The Michaelis-Menten plots and the nanomolar-range inhibition constants of compounds BB1 and 11 indicates that they are competitive inhibitors of AEP. Since BB1 contains a thiol group, it can form a disulfide bond with the active-site cysteine of AEP. The reactive group of compound 11 remains unclear, so to characterize its mode of inhibition further, progress curves were measured at increasing concentrations of inhibitor. The resulting curvilinear plots indicate that compound 11 inhibits AEP in a time and concentration-dependent manner (FIG. 6C). Additionally, plotting the pseudo-first-order rate constants of inhibition, k_{obs}, which were determined from the progress curves, yielded a hyperbolic curve, consistent with a two-step mechanism of inactivation (FIG. 6D). The rate of inactivation, k_{inact}, was found to be 0.075±0.002 min⁻¹, thus the second-order rate constant, k_{inact}/K_{I} is 1.2×10⁶ min⁻¹·M^{-31 1} suggesting that compound 11 irreversibly inactivates AEP. Compound BB1 also displays the progress curves indicative of a slow-binding inhibitor (FIG. 6E). However, the k_{obs} versus inhibitor concentration plot yielded a straight line, maybe due to the rapid disulfide formation reaction (FIG. 6F). The second-order rate constant of inhibition, k_{inact}/K_{I}, can be obtained from the slope of this curve and is 3.3×10⁶ min⁻¹·M⁻¹ and for compound BB1 indicating that this compound can form a disulfide linkage with the active-site thiol of AEP.

### Efficacy in Cellular Models of Acidosis

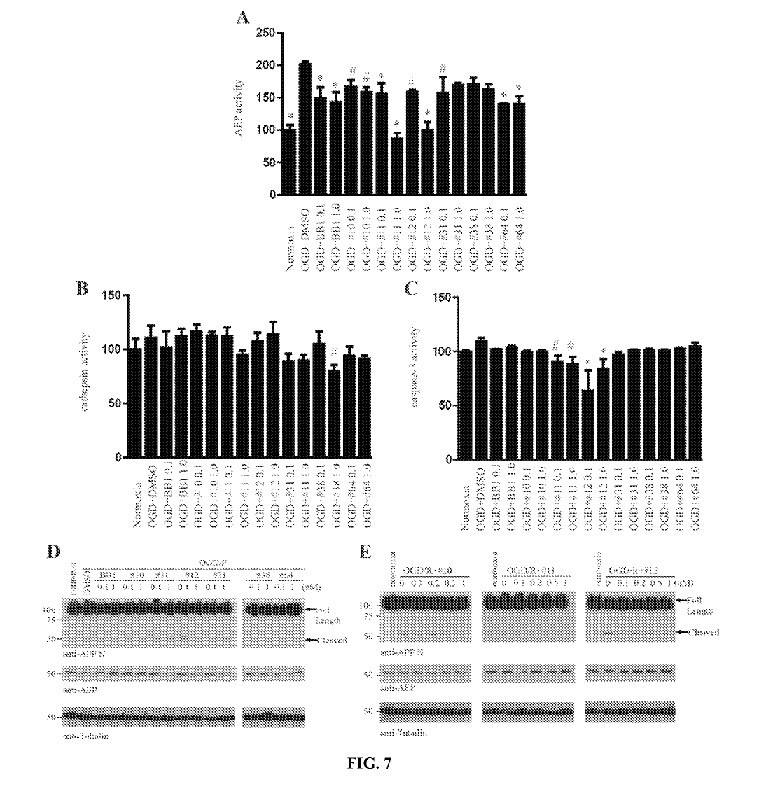

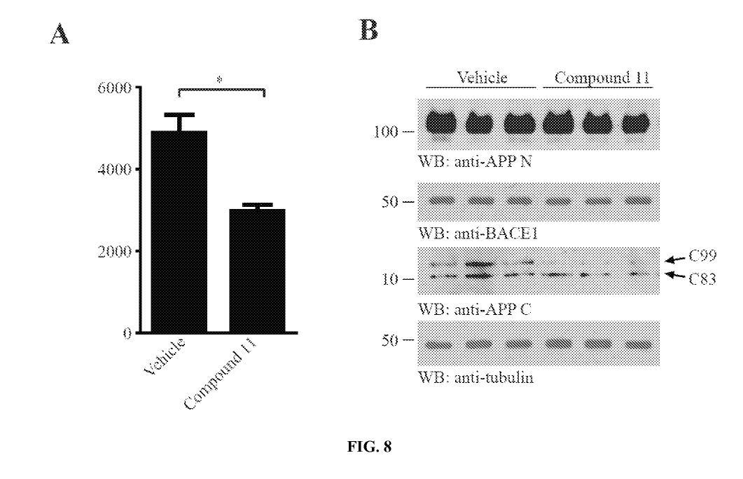

Depriving cells of oxygen and glucose is a cellular model that is employed to trigger acidosis in cultured cells. The efficacy of the compounds in a cellular model of oxygen-glucose deprivation (OGD) was determined in an effort to mimic the effects of stroke in primary cultured neurons. As a result of depriving neurons of oxygen and glucose-containing medium, AEP activity was doubled (FIG. 7A, ‘DMSO-Norm’ compared to ‘DMSO-OGD’), while Caspase-3 and Cathepsin activities remained relatively unchanged. In the presence of compound 11, there was a marked dose-dependent decrease in AEP activity, which was not observed for either Caspase-3 or Cathepsin. Compound 12 was also able to produce a slight decrease in AEP activity, selective to only that enzyme (FIG. 7). Since ischemia has been found to highly increase the risk of Alzheimer's Disease following stroke, the cleavage of the amyloid precursor protein (APP) was assess following OGD treatment (FIG. 7D and E). Cleavage of APP is observed in response to only DMSO treatment (FIG. 7D, lane 2), and it appears that upon the treatment of 1 μM of compounds 10, 11, and 12, and just 0.1 μM of compounds 31, 38 and 64, the cleavage was blocked, presumably due to AEP inhibition. FIG. 7E also shows a dose-dependent decrease of APP cleavage in response to the presence of compounds 10 and 12.

### Compound 11 has Neuroprotective Effects and Improves Cognitive Behavior in AD Mice

The acute and chronic toxicity of compound 11 was assessed by administering 100 mg/kg of the drug to C57BL/6J mice over a 30-day period via oral gavage. Over the full treatment time, no significant change in body weight was observed. Additionally, immunohistochemical staining demonstrated that there were no major differences between vehicle-treated and drug-treated animals. A complete blood count (CBC) analysis was also performed on the mice treated with compound 11 and it validates that all parameters are within the normal range of a healthy mouse. All together, these data demonstrate that compound 11 does not exhibit systemic toxicity in mice at the dosage of 100 mg/kg. Compound 11 possesses a very favorable ADMET profile with prominent blood-brain barrier permeability.

### Compound 11 Inhibits AEP Activity and Reduces APP Cleavage by BACE in a Mouse Model of AD

Active AEP cleaves APP and promotes the generation of Aβ. To test whether oral administration of compound 11 can attenuate the production of Aβ in vivo, 5XFAD mice were treated with compound 11 or vehicle for 3 months beginning at 2 months of age. 5XFAD mice coexpress a total of five mutations associated with familiar AD, and develop cerebral amyloid plaques at an early age. AEP activity assay showed that the oral compound 11 significantly inhibited the activity of AEP in mice brain (FIG. 8A). Blockade of AEP diminishes the subsequent BACE activity in cleaving APP. The production of C99, which is the C-terminal fragment of APP generated by β-site cleavage decreased after compound 11 treatment, but the expression of α-site cleavage product C83 was not altered by compound 11. Compound 11 treatment did not change the protein level of total APP or β-secrease (BACE1) (FIG. 8B, 2nd and 3rd panels). These results indicate that compound 11 inhibits the activity of AEP in the brain, and the inhibition of AEP activity decreases the β-site cleavage of APP and the production of Aβ. Hence, Compound 11 inhibits AEP and decreases APP degradation by BACE in AD mouse model.

### Chronic Oral Administration of Compound 11 Rescues Memory Deficits in 5XFAD Mice

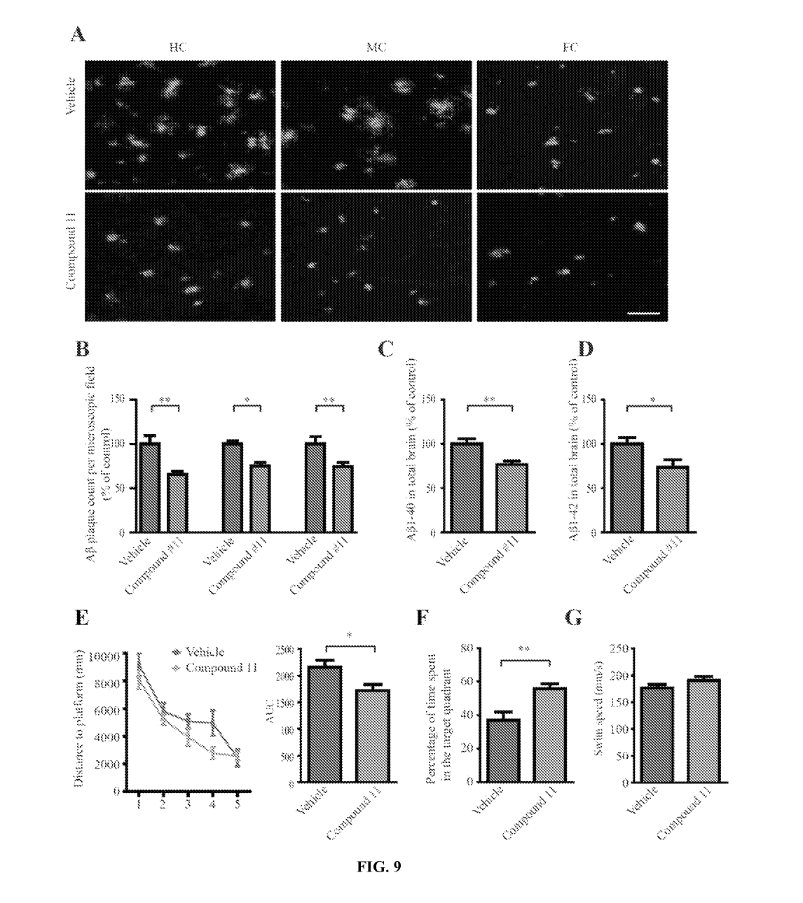

To investigate whether chronic oral administration of compound 11 can reverse the cognitive deficits of 5XFAD mice, the 3 months old mice we treated with Compound 11 via oral administration consecutively once daily for 3 months. Thioflavin S staining found that vehicle-treated 5XFAD mice have significant plaque deposition in the hippocampus (HC), motor cortex (MC), and frontal cortex (FC), which was attenuated by compound 11 (FIG. 9A, B). This result was confirmed by immunohistochemistry staining of amyloid plaque using an anti-Aβ antibody. Furthermore, compound 11 decreased the concentration of Aβ 1-40 and Aβ 1-42 in the brain lysates (FIG. 9C, D). To investigate the therapeutic efficacy of compound 11 on the cognitive activities, we assessed spatial memory formation using Morris water maze test. In the 5 acquisition days, the mice treated with compound 11 showed decreased distance to platform when compared with the vehicle-treated mice, indicating improved spatial learning (FIG. 9E). On the probe test, the mice treated with compound 11 spent more time in the target quadrant that formerly contained the platform, demonstrating rescue of spatial memory recall by compound 11 (FIG. 9F). The swim speed was not affected by compound 11 (FIG. 9G). These results indicate chronic treatment with compound 11 ameliorates memory deficits in 5XFAD model.

### Compound 11 Prevents Synaptic Loss and Restores Synaptic Plasticity in 5XFAD Mice

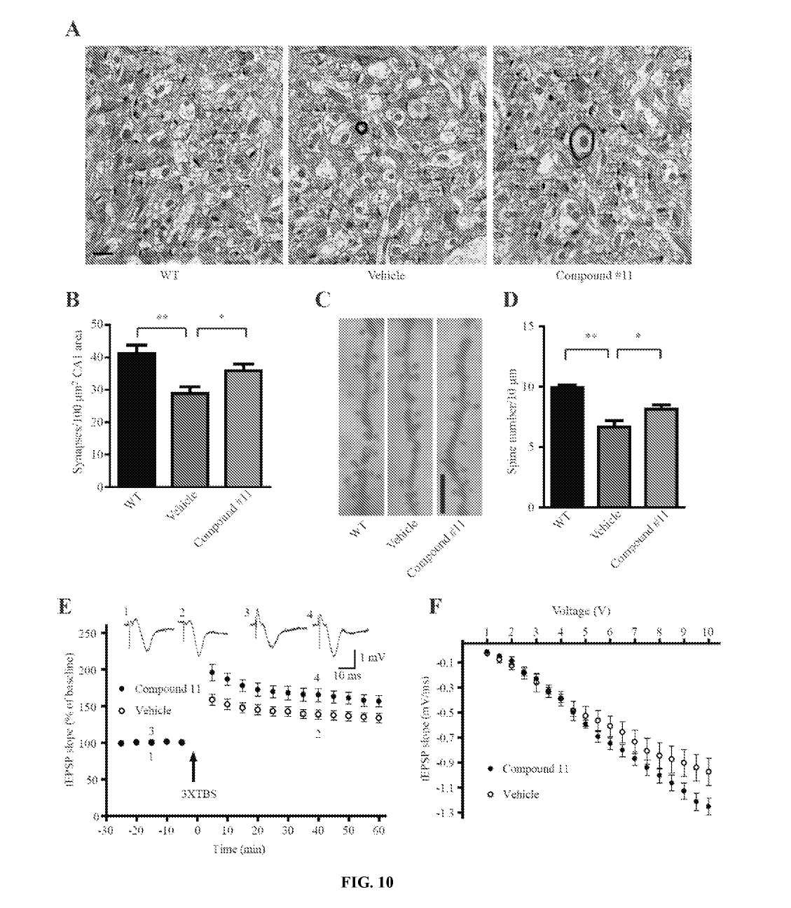

Synaptic dysfunction is the early feature of AD and is believed to be the basis of cognitive impairment. 5XFAD mice show decreased synaptic density compared to non-transgenic control mice. To investigate the effect of compound 11 on the synaptic dysfunction, the synaptic density in the CA1 area was assessed by electron microscopy. Compound 11 notably increased the density of synapse (FIG. 10A, B). The density of dendritic spines along individual dendrites of pyramidal neurons was assessed by Golgi stain. Again, compound 11 increased the density of spines (FIG. 10C, D). Long-term potentiation (LTP) is a measure of synaptic plasticity that underlies learning and memory. 5XFAD mice have decreased LTP magnitude at Schaffer collateral-CA1 pathways. Compound 11 treatment significantly reversed the LTP deficits in 5XFAD mice, indicating restoration of synaptic function by compound 11 (FIG. 10E, F).

### Methods

### Mice, Cells and Reagents

5XFAD mice were from Jackson lab, and were bred in a pathogen-free environment in accordance with Emory Medical School guidelines. The mice receive gavage treatments with vehicle or compound #11 at a dose of 10 mg/Kg/d. Anti-APP, anti-APP C, Mouse monocloncal Anti-APP N585 was developed using peptide NH₂-IKTEEISEVC-COOH and purified from Protein G affinity column. TUNEL In Situ cell death detection Kit was from Roche (Indianapolis, Ind.). Compound #11 was purchased from TCI (Portland, Oreg.). Chemicals were also purchased from Sigma-Aldrich. Asparagine Endopeptidase (AEP, Legumain) was obtained from Sino Biological, Cathepsin-S was obtained from Athens Research and Technology, Caspase-3 and Caspase-8 were obtained from Millipore. Pala cells were a gift from Dr. Colin Watts and were maintained in RPMI-1640 medium, supplemented with 10% FBS, 2 mg/mL glutamine, 100 U penicillin/streptomycin at 37° C., 5% CO₂ in a humidified incubator.

### High-Throughput Screening

In a micro-high throughput screening format, an Asinex compound library was screened for potential AEP inhibitors. In 1536-well plates, 625 mg/mL wild-type mouse kidney lysates was incubated with 16.7 μM library compound and read by an Envision Multilabel plate reader (Ex λ=360 nm, Emλ=460 nm) to obtain a background reading. 1 μM Cbz-Ala-Ala-Asn-AMC was added to initiate the reaction and after 15 min, the fluorescence was measured again and the background was subtracted from the final product. The percentage of inhibition, as compared to control wells, was determined and the top 736 compounds were subjected to dose-response confirmatory screening with the kidney lysates from AEP knockout mice and with 50 nM pure active AEP (Sino Biological).

### Enzymatic Assays

Cathepsin-S—100 nM enzyme was pre-incubated with inhibitor in assay buffer (100 mM NaH₂PO₄ pH 6.5, 100 mM NaCl) for 10 min at 37° C. The reaction was initiated upon addition of 25 μM substrate (Boc-Val-Leu-Lys-AMC (Bachem)).

Cathepsin-L—100 nM enzyme was pre-incubated with inhibitor in assay buffer (100 mM Sodium Acetate pH 5.5, 1 mM EDTA) for 10 min at 37° C. The reaction was initiated upon addition of 25 μM substrate (D-Val-Leu-Lys-AMC (Bachem)).

Caspase-3 and 8 —The enzyme was pre-incubated with inhibitor in assay buffer (100 mM HEPES pH 7.4, 0.1% CHAPS, 10% Sucrose) for 10 min at 37° C. The reaction was initiated upon addition of 25 μM substrate (Ac-Asp-Glu-Val-Asp-AMC (Bachem)).

AEP—50 nM enzyme was pre-incubated with inhibitor in assay buffer (50 mM Sodium Citrate pH 5.5, 0.1% CHAPS, 60 mM Na₂HPO₄, 1 mM EDTA) for 10 min at 37° C. The reaction was initiated upon addition of 10 μM Cbz-Ala-Ala-Asn-AMC (Bachem)).

### IC₅₀ Assays in Intact Pala Cells

Human B Lymphoblastoid Pala cells, which express high concentrations of AEP, were cultured in RPMI-1640, supplemented with 10% FBS, 2 mM L-Glutamine, 100 units/mL Penicillin, 100 μg/mL Streptomycin. Various concentrations of each compound were incubated with the Pala cells for 2 hr at 37° C. Cells were collected, washed twice with PBS and lysed in Lysis Buffer (20 mM Citric Acid, 60 mM Disodium Hydrogen Orthophosphate, 1 mM EDTA, 0.1% CHAPS, pH 5.8). The protein concentrations were normalized by Bradford assay and lysates were assayed with 5 μ,M Cbz-Ala-Ala-Asn-AMC. IC₅₀ values were determined by fitting the data to the equation: Fractional Enzymatic Activity=1/(1+([I]/IC₅₀)), in which [I] =Inhibitor concentration and IC₅₀ =inhibitor concentration that yields half-maximal activity. Data were analyzed with GraFit version 5.0.11 software package.

### Comet Assay (Single Cell Gel Electrophoresis)

Human hepatocellular carcinoma HepG2 cells were used to determine the genotoxicity of the compounds. The Comet Assay was performed according to the protocol provided in the Trevigen Kit (4250-050-K). Briefly, cells were pre-treated for 24 hrs with vehicle control or 50 μM compound. Cells were harvested, embedded in low-melt agarose and submerged in Lysis Buffer for 45 min at 4° C. After incubation in Alkaline Unwinding Solution (300 mM NaOH, 1 mM EDTA) for 20 min, the cells were subjected to electrophoresis in Alkaline Unwinding Solution at 300 mA for 30 min. Slides were washed with 70% ethanol, dried and stained with SYBR Green for 30 min at room temp. Nicked DNA was measured as percentage of tail DNA. One hundred comets were scored for each sample. See Kozics et al., (2011) Structure of flavonoids influences the degree inhibition of Benzo(a)pyrene-induced DNA damage and micronuclei in HepG2 cells, Neoplasma 58, 516-524.

### Micronucleus Assays

HepG2 cells were treated with vehicle or 50 μ.1\4 compound for 24 hours. Cells were washed with PBS, then incubated at 1:19 in a hypotonic solution (0.075 M KC1/0.9% NaCl) for 10 min at 37° C. Next, the cells were fixed with methanol:glacial acetic acid (3:1) for 15 min at 37° C., then rinsed and dried. Cells were stained with DAPI (2 mg/mL) for 30 min in the dark at room temp, rinsed with water, dried and mounted with glycerol. One thousand cells per dish were analyzed for each experiment; three independent experiments were performed.

### DTT Reversibility Assays

5× the approximate IC₅₀ value for each compound was added to the AEP reaction, as described above and allowed to react for 15 min. Simultaneously, 10 mM (final concentration) dithiothreitol (DTT) or L-cysteine was added to each reaction and incubated for an additional 15 min. At the end of the second 15 min incubation time, the fluorescence was measured for each sample. The amount of product formed in the presence of DMSO was used to determine the percentage of AEP activity in the presence of each drug.

### Oxygen-Glucose Deprivation

Compounds were pre-incubated with primary culture neurons, DIV13, for 30 min. The medium was exchanged for glucose-free DMEM and neurons were de-gased and incubated at 37° C., 95% N₂/5% CO₂ for 4 hrs with compounds. The medium was exchanged for DMEM and supplemented with compounds, then neurons were reperfused for 18 hrs under normoxic conditions. The neuronal lysates were prepared for AEP, Caspase-3 and Cathepsin assays. Moreover, immunoblotting analysis was conducted with the neuronal lysates using anti-APP, anti-AEP antibodies.

### Enzyme Inhibition Assays

To determine the inhibition constants and the mechanism by which compounds BB1, 11 and 38 inhibit AEP, the steady-state kinetic parameters for the hydrolysis of the peptide substrate, Z-AAN-AMC, was determined in the absence or presence of increasing concentrations of inhibitor. In these assays, specified concentrations of the inhibitor were pre-incubated with substrate for 10 min at 37° C., then 50 nM AEP was added to initiate the reaction, which was quenched after 10 min. The RFU values of the reaction product were converted to micromolar values with an AMC standard curve and the final reaction rates were plotted against substrate concentration and globally fit to equations representative of competitive inhibition (eq 1), noncompetitive inhibition (eq 2), mixed inhibition (eq 3) and uncompetitive inhibition (eq 4) using a nonlinear least fit squares approach by GraFit version 5.0.11.

ν=Vₘₐₓ[S]/([S]+Kₘ(1+[1]/Kᵢₛ)) (eq 1),

ν=Vₘₐₓ[S]/([S](1+[1]/Kₗ)+Kₘ(1+[1]/Kᵢ)) (eq 2),

ν=Vₘₐₓ[S]/([S](1+[1]/Kᵢᵢ)+Kₘ(1−[1]/Kᵢₛ)) (eq 3),

ν=Vₘₐₓ[S]/([S](1+[1]/Kᵢᵢ)+Kₘ) (eq 4).

In the equations, Kᵢᵢ is the intercept Kᵢ, and Kᵢₛ is the slope Kᵢ. The mode of the inhibition induced by the compounds on AEP was determined by the best fit of the data to equations 1 -4. Visual inspection of the fits, and a comparison of the standard errors, was used to confirm these assignments.

### Time Course Inactivation Assays

Progress curves were generated by incubating 5 μM Z-AAN-AMC and the specified concentration of inhibitor in assay buffer at 37° C. for 10 min. The reaction was initiated by the addition of 50 nM AEP and quenched after 10 min. The concentration of the product was determined from an AMC standard curve and the data was fit by nonlinear regression. Since the curves were nonlinear, they were fit to equation 5, using the GraFit version 5.0.11 software package,

[Product]=νᵢ(1−e^{-kobs.app*t})/k_{obs.app} (eq 5),

where νᵢ is the initial velocity, k_{obs.app} is the apparent pseudo-first order rate constant for inactivation, and t is time. Equation 6,

k_{obs}=((1+[S])/Kₘ)k_{obs.app} (eq 6),

was used to correct the apparent pseudo-first-order inactivation rate constants, obtained from this analysis, for substrate concentration and the pseudo-first-order inactivation rate constants, i.e. k_{obs}, thus obtained, were plotted against the tested inhibitor concentrations. As the data are consistent with a two-step mechanism of inactivation, they were fit to equation 7,

k_{obs}=(k_{inact}[1])/(K_{f}+[1]) (eq 7),

using the GraFit version 5.0.11 software, where K_{I} is the concentration of inactivator that yields half-maximal inactivation, k_{inact} is the maximal rate of inactivation, and [I] is the concentration of inactivator.

### Electron Microscopy

Synaptic density was determined by electron microscopy as described previously (22). After deep anesthesia, mice were perfused transcardially with 2% glutaraldehyde and 3% paraformaldehyde in PBS. Hippocampal slices were post-fixed in cold 1% OsO₄ for 1 h. Samples were prepared and examined using standard procedures. Ultrathin sections (90 nm) were stained with uranyl acetate and lead acetate and viewed at 100 kV in a JEOL 200CX electron microscope. Synapses were identified by the presence of synaptic vesicles and postsynaptic densities.

### Mice Brain Tissue Preparation and Protein Extraction

After completion of the behavioral test, mice were deeply anaesthetized with pentobarbital and transcardially perfused with saline, and the brains were rapidly removed. One hemisphere was fixed in 4% phosphate-buffered paraformaldehyde, while the other was snap frozen for biochemical analysis. For brain protein extraction, hemispheres were first extracted in RIPA buffer (25 mM Tris-HCl, pH 7.5, 150 mM NaCl, 1% NP 40, 0.5% NaDOC, 0.1% SDS), centrifuged at 100,000 rpm for 30 min and the pallet containing insoluble Aβ was further extracted in 2% SDS, 25 mM Tris-HCl, pH 7.5.

### Electrophysiological Analysis

Electrophysiological analysis was carried out as previously described (23). Briefly, vehicle—and compound #11-treated 5XFAD mice were anaesthetized with isoflurane, decapitated, and their brains dropped in ice-cold artificial cerebrospinal fluid (a-CSF) containing 124 mM NaCl, 3 mM KCl, 1.25 mM NaH₂PO₄, 6.0 mM MgCl₂, 26 mM NaHCO₃, 2.0 mM CaCl₂, and 10 mM glucose. The hippocampi were cut into 400-μm thick transverse slices with a vibratome. After incubation at room temperature (23-24° C.) in a-CSF for 60-90 min, slices were placed in a recording chamber (RC-22C, Warner Instruments) on a stage of an up-right microscope (Olympus CX-31) and perfused at a rate of 3 ml per min with a-CSF (containing 1 mM MgCl₂) at 23-24° C. A 0.1 MΩ tungsten monopolar electrode was used to stimulate the Schaffer collaterals. The field excitatory post-synaptic potentials (fEPSPs) were recorded in CA1 stratum radiatum by a glass microelectrode filled with a-CSF with resistance of 3-4 MEI The stimulation output (Master-8; AMPI, Jerusalem) was controlled by the trigger function of an EPC9 amplifier (HEKA Elektronik, Lambrecht, Germany). fEPSPs were recorded under current-clamp mode. Data were filtered at 3 kHz and digitized at sampling rates of 20 kHz using Pulse software (HEKA Elektronik). The stimulus intensity (0.1 ms duration, 10-30 μA) was set to evoke 40% of the maximum f-EPSP and the test pulse was applied at a rate of 0.033 Hz. LTP of fEPSPs was induced by 3 theta-burst-stimulation (TBS), it is 4 pulses at 100 Hz, repeated 3 times with a 200-ms interval). Paired-pulse facilitation (PPF) was examined by applying pairs of pulses, which were separated by 20-500 ms intervals. The magnitudes of LTP are expressed as the mean percentage of baseline fEPSP initial slope.

### Western Blot Analysis

The mouse brain tissue or human tissue samples was lysed in lysis buffer (50 mM Tris, pH 7.4,40 mM NaCl, mM EDTA, 0.5% Triton X-100, 1.5 mM Na₃VO₄, 50 mM NaF, 10 mM sodium pyrophosphate, 10 mM sodium β-glycerophosphate, supplemented with protease inhibitors cocktail), and centrifuged for 15 min at 16,000 g. The supernatant was boiled in SDS loading buffer. After SDS-PAGE, the samples were transferred to a nitrocellulose membrane. Western blot analysis was performed with a variety of antibodies.

### Immunohistochemistry

Free-floating 30-μm-thick serial sections were treated with 0.3% hydrogen peroxide for 10 min, and then incubated with anti-AEP-cleaved APP (1:500) or anti-Aβ (1:500) overnight. The signal was developed using Histostain-SP kit (#956543, invitrogen) according to the manufacturer's instructions.

### Aβ Plaque Staining

Amyloid plaques were stained with Thioflavin-S. The deparaffinized and hydrated sections were incubated in 0.25% potassium permanganate solution for 20 min, rinsed in distilled water, and incubated in bleaching solution containing 2% oxalic acid and 1% potassium metabisulfite for 2 min. After rinsed in distilled water, the sections were transferred to blocking solution containing 1% sodium hydroxide and 0.9% hydrogen peroxide for 20 min. The sections were incubated for 5 s in 0.25% acidic acid, then washed in distilled water and stained for 5 min with 0.0125% Thioflavin-S in 50% ethanol. The sections were washed with 50% ethanol and placed in distilled water. Then the sections were covered with glass cover using mounting solution.

### Aβ42 ELISA

The mice brains were homogenized in 8X mass of 5 M guanidine HCl/50 mM Tris HCl (pH 8.0), and incubated at room temperature for 3 h. Then the samples were diluted with cold reaction buffer (phosphate buffered saline with 5% BSA and 0.03% Tween 20, supplemented with protease inhibitor cocktail), and centrifuged at 16 000 g for 20 min at 4° C. The supernatant were analysed by human Aβ42 ELISA kit according to the manufacturer's instructions (KHB3441, Invitrogen). The Aβ42 concentrations were determined by comparison with the standard curve.

### Morris Water Maze

Female wild-type and 5XFAD mice maintained on standard drinking water or compound #11 were trained in a round, water-filled tub (52 inch diameter) in an environment rich with extra maze cues. An invisible escape platform was located in a fixed spatial location 1 cm below the water surface independent of a subjects start position on a particular trial. In this manner, subjects needed to utilize extra maze cues to determine the platform's location. At the beginning of each trial, the mouse was placed in the water maze with their paws touching the wall from 1 of 4 different starting positions (N, S, E, W). Each subject was given 4 trials/day for 5 consecutive days with a 15-min inter-trial interval. The maximum trial length was 60 s and if subjects did not reach the platform in the allotted time, they were manually guided to it. Upon reaching the invisible escape platform, subjects were left on it for an additional 5 s to allow for survey of the spatial cues in the environment to guide future navigation to the platform. After each trial, subjects were dried and kept in a dry plastic holding cage filled with paper towels to allow the subjects to dry off The temperature of the water was monitored every hour so that mice were tested in water that was between 22 and 25° C. Following the 5 days of task acquisition, a probe trial was presented during which time the platform was removed and the percentage of time spent in the quadrant which previously contained the escape platform during task acquisition was measured over 60 s. All trials were analysed for latency, swim path length, and swim speed by means of MazeScan (Clever Sys, Inc.).

### Golgi Staining

Mice brains were fixed in 10% formalin for 24 h, and then immersed in 3% potassium bichromate for 3 days in the dark. The solution was changed each day. Then the brains were transferred into 2% silver nitrate solution and incubate for 24 h in the dark. Vibratome sections were cut at 60 μm, air dried for 10 minutes, dehydrated through 95% and 100% ethanol, cleared in xylene and coverslipped. For measurement of spine density, only spines that emerged perpendicular to the dendritic shaft were counted.

What is claimed is:

1. A compound of the following formula: 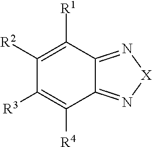
prodrugs, derivatives, or salts thereof wherein,
X is O or s;
R1 is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R1 is optionally substituted with one or more, the same or different, R10;
R10 is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R10 is optionally substituted with one or more, the same or different, R11;
R11 is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl,N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-m ethyl sulfamoyl, N-ethyl sulfamoyl, N,N-dimethylsulfamoyl, N,N-diethylsulfamoyl, N-methyl-N-ethylsulfamoyl, carbocyclyl, aryl, and heterocyclyl;
R2 is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R2 is optionally substituted with one or more, the same or different, R20;
R20 is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R20 is optionally substituted with one or more, the same or different, R21;
R21 is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl,N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methyl sulfamoyl, N-ethyl sulfamoyl, N,N-dimethylsulfamoyl, N,N-diethylsulfamoyl, N-methyl-N-ethylsulfamoyl, carbocyclyl, aryl, and heterocyclyl;
R3 is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R3 is optionally substituted with one or more, the same or different, R30;
R30 is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R30 is optionally substituted with one or more, the same or different, R31;
R31 is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl,N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methyl sulfamoyl, N-ethyl sulfamoyl, N,N-dimethylsulfamoyl, N,N-diethylsulfamoyl, N-methyl-N-ethylsulfamoyl, carbocyclyl, aryl, and heterocyclyl;
R4 is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R4 is optionally substituted with one or more, the same or different, R40;
R40 is selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R40 is optionally substituted with one or more, the same or different, R41; and
R41 is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl,N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methyl sulfamoyl, N-ethyl sulfamoyl, N,N-dimethylsulfamoyl, N,N-diethylsulfamoyl, N-methyl-N-ethylsulfamoyl, carbocyclyl, aryl, and heterocyclyl.

2. The compound of claim 1, wherein R1 is amino.

3. The compound of claim 1, wherein R2 is hydrogen.

4. The compound of claim 1, wherein R3 is hydrogen.

5. The compound of claim 1, wherein R4 is a heterocyclyl.

6. The compound of claim 1, wherein the compound is 7-morpholinobenzo[c][1,2,5] oxadiazol-4-amine.

7. A pharmaceutical composition comprising a compound of claim 1 and a pharmaceutically acceptable excipient.

8. The pharmaceutical composition of claim 7 in the form of a pill, capsule, tablet, or saline aqueous buffer.

9. The pharmaceutical composition of claim 7, wherein the pharmaceutically acceptable excipient is selected from a saccharide, disaccharide, sucrose, lactose, glucose, mannitol, sorbitol, polysaccharides, starch, cellulose, microcrystalline cellulose, cellulose ether, hydroxypropyl cellulose (HPC), xylitol, sorbitol, maltito, gelatin, polyvinylpyrrolidone (PVP), polyethylene glycol (PEG), hydroxypropyl methylcellulose (HPMC), crosslinked sodium carboxymethyl cellulose, dibasic calcium phosphate, calcium carbonate, stearic acid, magnesium stearate, talc, magnesium carbonate, silica, vitamin A, vitamin E, vitamin C, retinyl palmitate, selenium, cysteine, methionine, citric acid, and sodium citrate, methyl paraben, propyl paraben, and combinations thereof.

10. A method of treating or preventing a neurodegenerative disease or condition comprising administering an effective amount of a pharmaceutical composition of claim 7 to a subject in need thereof.

11. The method of claim 10, wherein the neurodegenerative disease or condition is Alzheimer's Disease, stroke, traumatic brain injury, seizure, cognitive impairment.
